(19) United States
(12) Patent Application Publication
(10) Pub. No.: US 2003/0044965 A1
(43) Pub. Date: Mar. 6, 2003

(54) Long term preservation and storage of viable dried bacteria

(76) Inventors: Alfred Mateczun, Bethesda, MD (US); Leonard Peruski, Chevy Chase, MD (US)

Correspondence Address: Naval Medical Research Center, Office of Counsel (Code 00L), 503 Robert Grant Avenue, Silver Spring, MD, 20910-7500, US

(21) Appl. No.: 10/108,344

(22) Filed: Mar. 29, 2002

Related U.S. Application Data
(63) Continuation-in-part of application No. 09/159,568, filed on Sep. 24, 1998, now abandoned.

(51) Int. Cl. C12N 1/04; A01N 63/00; C12N 1/36

(52) U.S. Cl. 435/260; 424/93.4; 424/93.462; 424/93.47; 424/93.48

(57) ABSTRACT

Live bacteria are preserved by subjecting drying an aqueous system containing the growing bacteria at room temperature, without special equipment, in the presence of trehalose with or without the addition of divalent cations as stabilizing agents.

### CROSS-REFERENCE TO RELATED APPLICATION(S)

This application is a continuation-in-part of U.S. Ser. No. 09/159,568, filed on Sep. 24, 1998 and claims the benefit of the filing date thereof.

### SPECIFICATION

Field of the Invention

The maintenance of live bacteria in a stable form is critical for the conduct of basic microbiological studies and the development and production of diagnostic assays and vaccines. Furthermore, transport of bacterial strains between research or production facilities often requires the organisms to be in a preserved state to prevent damage or genetic alteration. Several methods exist for the preservation of bacterial cells. Two widely used methods are freeze-drying (Crowe and Crowe 1991; Crowe et al., 1992) and desiccation (Carpenter et al., 1987 and 1988; Oliver et al., 1995; Roser, 1991; Shier, 1988). Methods for the freeze-drying of bacteria have been relatively successful. Because successfully freeze-dried cells can be stored in the absence of freezing conditions, these procedures are more convenient and less expensive compared to super-cooled systems such as with liquid nitrogen or ultralow temperature freezers. Cryoprotectants are included when cells are frozen in order to prevent the damaging effects of water crystals. Similarly, desiccation requires anhydroprotectants to prevent destruction of bio-molecules as the water is removed during the preservation process.

Some anhydrobiotic organisms are actually able to survive in a nearly completely dry or desiccated state without freezing. Members of the tardigrades, for example, are capable of surviving under these conditions (Crowe and Cooper, 1971). These organisms are highly complex, with heads, limbs and internal body parts similar to those of insects. Anhydrobiosis is made possible, in large part, through the elaboration and distribution of a sugar, trehalose, which supports cellular membrane structure against collapse by substituting for water at the polar head groups of the lipids (Crowe and Crowe, 1991, Crowe et al., 1992; Leslie et al., 1995; Mansure et al., 1994). Further studies have suggested that trehalose has cryoprotective properties (Crowe et al., 1992; Israeli et al., 1993; Leslie et al., 1994).

Chemically, trehalose is a non-reducing disaccharide consisting of two linked glucose molecules and has approximately half the sweetness of sucrose. Empirical evidence indicates that high concentrations of trehalose in the tissues of certain insects and desert plants allows them to survive in a state of suspended animation under conditions of water deficiency (Hirsh, 1987). It has also been suggested that trehalose is an important factor in the survival of frogs during the winter months in northern climates (Lee et al., 1992).

U.S. Pat. No. 5,149,653 describes the use of trehalose in the preservation of viruses. Unlike mammalian cells, however, live virus vaccines cannot be easily frozen without loosing their immunogenic effect. Therefore, they must either be kept in aqueous media under cool sterile conditions such as in a refrigerator or stored at room temperature in the presence of preserving agent such as trehalose.

Depending on the species, bacteria can be stored either at room temperature, refrigerated as slant cultures on nutrient agar, or frozen. Storage of bacteria on agar, for those species that will tolerate it, is relatively convenient. However, it has been shown that organisms can genetically alter over time, especially the genetic material that is carried on plasmids. Additionally, not all bacteria can be stored for long periods on nutrient agar. Although most bacteria can be frozen, like viruses and eukaryotic cells, some alterations to the bacteria can occur upon thawing. Furthermore, recovery rates of bacteria are variable among species and among freezing conditions. Also, the process for freezing of cells is often relatively complex, requiring either super-cooled systems such as liquid nitrogen or mechanical freezers. In some circumstances, especially field conditions or operations studies conducted in developing countries, the availability and maintenance of super-cooled systems or mechanical freezers, or even refrigerators, is often problematic.

Previous studies have reported the use of trehalose in the preservation of mammalian cells by lyophilization or, in the case of viral viruses by evaporation at ambient temperatures. The use of trehalose for the preservation of bacteria, which have distinct membrane structure compared to mammalian cells or viruses has not been previously described. Although many strains of bacteria can withstand freezing and drying in the absence of a special preserving agent, because of the presence of a rigid outer wall, the efficiency of recovery is often poor. Many more fragile bacteria are incapable of being preserved without cryoprotective additives. Also, successful preservation of bacterial cells requires that thawed cells retain the ability to reproduce. Thus, the ability of a method or compound to preserve mammalian cells, particularly non-reproducing mammalian cells such a platelets and red blood cells, does not predict the ability of that method or compound to preserve bacterial cells. Here we describe a simple method for long-term preservation of gram-negative and gram-positive bacteria by desiccation at ambient temperature in the presence of trehalose and divalent cations.

### SUMMARY OF THE INVENTION

Accordingly, an object of this invention is to provide a new, effective and economical process for the preservation of bacterial species without requiring special equipment beyond what is typically found in microbiology laboratories.

An additional object is that the bacteria can be easily reconstituted with a high rate of survival and with the ability to reproduce.

A further object is that the bacteria retain characteristics and are not detectably altered after freezing and reconstitution.

These and additional objects of the invention are accomplished by drying gram-negative and gram-positive bacteria in the presence of the cryopreservative trehalose and more preferably in the presence of certain divalent cations. The method of preservation involves drying bacteria in the presence of specific cryopreservation materials such that the organisms can be reconstituted in a viable form with little or no genetic damage. This process is capable of being used on a number of different bacterial genera and species, beyond those immediately described here. Following preservation and reconstitution the cells retain their genotypic and phenotypic characteristics. Although the process has been tested most with aerobic bacteria, it should also be capable of successful application to anaerobic bacteria. The invention has been successfully used for the preservation of a wide variety of bacteria, including, but not limited to Campylobacter jejuni, Escherichia coli, Staphylococcus aureus, Pseudomonas aeruginosa, and Vibrio cholerae.

### BRIEF DESCRIPTION OF THE DRAWINGS

A more complete appreciation of the invention will be readily obtained by reference to the following Description of the Preferred Embodiments and the accompanying figures.

FIG. 1 is a diagrammatic presentation of the preservation methods.

FIG. 2 shows the survival of various bacterial species after desiccation and rehydration according to the present invention.

### DESCRIPTION OF THE PREFERRED EMBODIMENTS

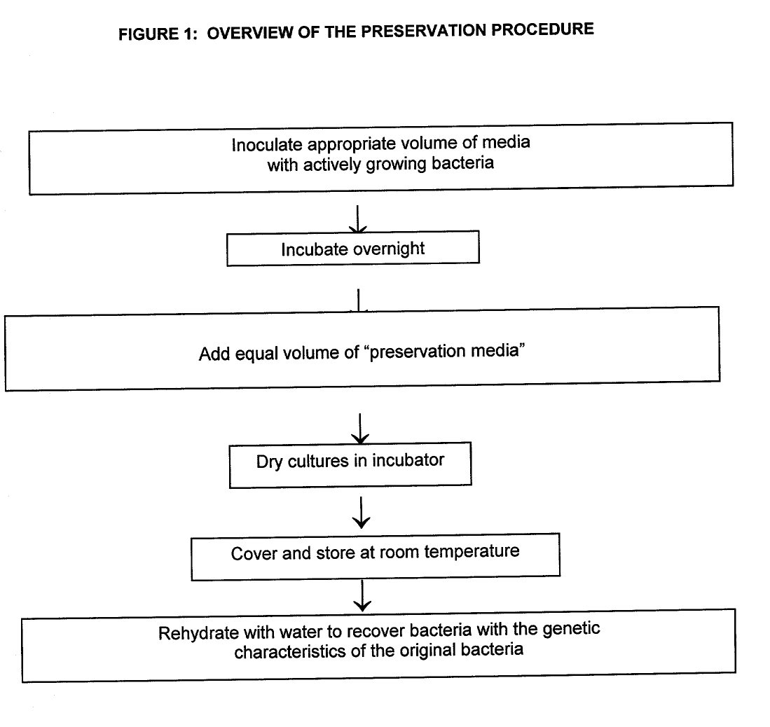

The present invention can be utilized to preserve for short or long-term storage or for transport, a large number of different bacterial genera and species. The process is easy to conduct, relatively inexpensive and does not require equipment outside of that normally encountered in a standard microbiology laboratory. The process, illustrated in FIG. 1, entails inoculating bacteria in a small volume of bacterial culture media common in the art and described in Manual of Clinical Microbiology, ASM. The type of media selected is dependent on the species of bacteria to be preserved. Some preparatory procedures are employed, prior to preservation. These include: 1) growth of bacteria on suitable agar or liquid broth; 2) harvesting of organisms by scraping colonies off of agar plates or collection from liquid cultures; 3) centrifugation of bacteria and resuspension in bacterial culture media at an appropriate concentration for expansion for preservation the following day. The bacterial cell density at harvesting depends on the bacterial species, however typical densities are 0.1 to 0.5 OD units. The preservation procedure is carried out the following day. After resuspension the bacteria are added to wells of a 96 well, or other type of suitable plate, and incubated overnight. Subsequent to the overnight incubation an equal volume of “preservation solution”, containing from 10 mM to 200 mM trehalose with or without the addition of 1 to 10 mM one or more of the divalent cations Mg++, Ca++, 2n++, Mm++ (typically in forms such as CaCl₂, ZnCl₂, or MgCl₂) is added to the cultures, the cultures placed in an incubator, at 37° C. and allowed to dry completely over a period of up to 96 hours freezing is not involved. The dried cultures covered, sealed in protective bags or containers and stored at room temperature at 20° C. to 25° C.

Reconstitution of the bacteria is accomplished by the addition of sterile water equal to the original total volume, pre-warmed to growth temperatures (37° to 42° C.), depending on bacterial species being preserved, and then plated onto bacterial growth media.

Having described the invention, the following examples are given to illustrate specific applications of the invention including the best mode now known to perform the invention. These specific examples are not intended to limit the scope of the invention described in this application.

### EXAMPLE 1

Evaluation of the Effect of Trehalose on the Preservation of Bacterial Strains at Room Temperature in the Dry State.

1. Bacterial Strains

Bacteria strains with the following phenotypic characteristics were each tested for their ability to be preserved and reconstituted using trehalose as preservation agent: Escherichia coil (W3110 Wild-type), Vibrio cholerae (O139), Salmonella typhimurium LT2, and Shigella flexneri 2a.

2. Preservation

The different strains of bacteria listed above were each individually inoculated into standard culture media in wells of a 96-well microtiter dish. They were grown overnight at 37° C. The following day an equal volume of preservation media, with 10 mM of each divalent cation (CaCl₂, MgCl₂ ZnCl₂), containing increasing concentrations of trehalose was added to the growing cultures giving a final concentration of trehalose from 10 mM to 200. The cultures were gently rocked over a 96 hour period and the contents allowed to dry. The dish was then covered and placed at room temperature.

3. Reconstitution

After 72 hours, individual cultures were rehydrated with sterile water, pre-warmed to 37° C. and added to Luria-Broth media to test for viability. The results are shown in Table 1. As shown in Table 1, with the exception of E. coli, all the species of bacteria tested had a higher level of viability after 72 hours when preserved in the presence of trehalose than without.

TABLE 1

| | 10 mM each of CaCl₂, MgCl₂ and ZnCl₂ plus | | | | | |
|---|---|---|---|---|---|---|
| | | | | | | 200(mM |
| Bacterial Strain | 10 | 25 | 50 | 100 | 150 | Trehalose) |
| E. coli (wild-type) | + | + | + | + | + | + |
| Vibrio cholerae | − | +/− | + | + | + | + |
| Salmonella typhimurium | +/− | + | + | + | + | + |
| Shigella flexneri 2a | +/− | +/− | + | + | + | + |

### EXAMPLE 2

Evaluation of the genotypic and phenotypic characteristics of bacterial strains after cryopreservation with trehalose and the divalent cations; CaCl₂, MgCl₂, and ZnCl₂. ps 1. Bacterial Strains

E. coli strains with the following phenotypic characteristics were each tested for their ability to be preserved and reconstituted using trehalose as preservation agent: Wild-type, ETEC:LT, ETEC:ST, ETEC:LT/ST, Vibrio cholerae O139, Salmonella typhimurium LT2, Shigella flexneri 2a.

2. Preservation

The different strains of bacteria listed above were each individually inoculated into standard culture media in wells of a 96-well microtiter dish. They were grown overnight at 37° C. The following day an equal volume of preservation media containing increasing concentrations of trehalose was added to the growing cultures giving a final concentration of trehalose from 10 mM to 200. The cultures were gently rocked over a 96 hour period and the contents allowed to dry. The dish was then covered and placed at room temperature.

3. Reconstitution

At regular intervals, up to 120 days, individual cultures were rehydrated with sterile water, pre-warmed to 37° C. and added to brain-heart infusion broth (BHIB). The results of the bacterial viability are shown in Table 2 at 100 mM trehalose and 10 mM each of ZnCl₂, CaCl₂, and MgCl₂.

4. Phenotypic/Genotypic Analysis

After growing overnight in BHIB, the individual colonies were tested for their ability to grow on selective media, and for the presence of genetic markers by polymerase chain reaction. The colonies were also tested for identity by API20E biochemical test strips. The results of these analyses are shown in Table 2.

TABLE 2

| | Media | | | | |
|---|---|---|---|---|---|
| Organism | MAC | M9 | TCBS | API20E | PCR |
| E. coli W3110 (wild-type) | + | + | NA | + | NA |
| E. coli (ETEC:LT) | + | NA | NA | + | + |
| E. coli (ETEC:ST) | + | NA | NA | + | + |
| E. coli (ETEC:LT/ST) | + | NA | NA | + | + |
| Vibrio cholerae (0139) | NA | NA | + | + | + |
| Salmonella typhimurium (LT2) | + | NA | NA | + | + |
| Shigella flexneri 2a | + | NA | NA | + | + |

### EXAMPLE 3

Effect of Trehalose and Divalent Cations.

First, we compared the drying of bacteria in the presence or absence of trehalose. Trehalose was used at a single concentration of 200 mM. The bacteria tested were Escherichia coli, Vibrio cholerae, Salmonella typhimurium, Shigella flexneri, Bacillus subtilis, and Campylobacter jejuni. Briefly, cells were grown overnight in 1.5 ml of brain heart infusion (BHI) broth in 15 ml conical tube at 37° C., 200 RPM. The cells were mixed with an equal volume of trehalose/water solution or water by gentle mixing and dried over a period of one week at ambient temperature. After a week in the desiccated state, the cells were rehydrated with sterile distilled water and 10 μl of the rehydrated cells were inoculated into BHI broth and incubated overnight at 37° C. The results of these experiments are summarized in Table 3.

Table 3: Viability After Drying in the Presence or Absence of

TREHALOSE

| | RELATIVE VIABILITY | |
|---|---|---|
| BACTERIA | w-Trehalose | w/o-Trehalose |
| E. coli | + | + |
| V. cholerae | − | − |
| S. typhimurium | + | +/− |
| S. flexneri | + | +/− |
| B. subtilis | + | + |
| C. jejuni | − | − |

As a follow on to this experiment, cations were added a concentration that approximated that used to maintain the transformation competency of bacteria to ascertain if this modification would improve survivability of the more fastidious C. jejuni and V. cholerae. The concentration of trehalose was varied over the range from 0 to 200 mM, but the level of CaCl₂, MgCl₂, and ZnCl₂ remained fixed at a final concentration of 10 mM. Bacteria were dried at ambient temperature sterile polypropylene tubes in the presence or absence of trehalose. After 72 hours post-drying the bacteria were tested for viability by inoculation as before into BHI media. All tests were done in triplicate and the results are summarized in Table 4.

TABLE 4
EFFECT OF TREHALOSE CONCENTRATION ON VIABILITY OF
BACTERIA IN THE PRESENCE OF 10 mM EACH OF CaCl₂, MgCl₂ &
ZnCl₂

| | TREHALOSE CONCENTRATION (NM) | | | | | | | | |
|---|---|---|---|---|---|---|---|---|---|
| BACTERIA | 0 | 10 | 25 | 50 | 100 | 125 | 150 | 175 | 200 |
| E. coli | +/− | + | + | + | + | + | + | + | + |
| V. cholerae | − | − | +/− | + | + | + | + | + | + |
| S. typhimurium | − | +/− | + | + | + | + | + | + | + |
| S. flexneri | +/− | +/− | +/− | + | + | + | + | + | + |
| B. subtilis | + | + | + | + | + | + | + | + | + |
| C. jejuni | − | − | +/− | + | + | + | + | + | + |

Based on the above results, a solution composed of 100 mM trehalose, 10 mM CaCl₂, 10 mM MgCl₂, and 10 mM ZnCl₂ was selected for further investigation.

Effect of Temperature.

In this set of experiments the effect of drying temperature was examined on the preservation of E. coli. Five temperatures were tested: ambient (˜22° C.), 37° C., 42° C., 45° C., and 50° C. (lethal for the growth of E. coli). Using the prototype preservation solution described above, the bacteria were first grown at 37° C. in BHI broth, mixed with an equal volume of solution, and dried at the temperatures listed above. Three days later, the desiccated bacteria were rehydrated with sterile distilled water and 10 μl of the rehydrated cells were inoculated into BHI broth and incubated overnight at 37° C. The results of these experiments are summarized in the Table 5.

TABLE 5
EFFECT OF TEMPERATURE

| | RELATIVE VIABILITY | | |
|---|---|---|---|
| TEMPERATURE | Exp. 1 | Exp. 2 | Exp. 3 |
| Ambient | + | + | + |
| 37° C. | + | + | + |
| 42° C. | + | + | + |
| 45° C. | + | − | + |
| 50° C. | − | − | − |

From these experiments it was found that temperatures of 42° C. or lower were optimal for desiccation, closely mirroring the optimal temperature range for the growth of E. coli.

Evaluation of the Genotypic and Phenotypic Traits After Desiccation.

E. coli strains with the following phenotypic characteristics were each tested for their ability to be preserved and reconstituted using trehalose as preservation agent: Wild-type, ETEC:LT, ETEC:ST, ETEC:LT/ST. Each strain was individually inoculated into standard culture media in wells of a 96-well microtiter dish and grown overnight at 37° C. The following day an equal volume of preservation media containing increasing concentrations of trehalose was added to the growing cultures giving a final concentration of trehalose from ranging from 10 mM to 200. The cultures were gently rocked to mix the contents and allowed to dry. The dish was then covered and placed at room temperature. After 30 days in the desiccated state, the strains were rehydrated with sterile water, pre-warmed to 37° C., and added to brain-heart infusion broth (BHIB). After growing overnight in BHIB, the strains were tested for their ability to grow on selective media and for the presence of genetic markers by polymerase chain reaction. The colonies were also tested for identity by API20E biochemical test strips. The results of these analyses are shown in Table 6. There was no discernable change in the growth characteristics, biochemical test profile, or PCR analysis of genetic markers after desiccation and revival, demonstrating that this process does not affect either phenotypic or genotypic features.

TABLE 6
GENOTYPIC AND PHENOTYPIC ANALYSIS

| | Media | | Other | |
|---|---|---|---|---|
| Organism | MAC | M9 | API20E | PCR |
| E. coli W3110 | + | + | + | NA |
| E. coli (ETEC:LT) | + | NA | + | + |
| E. coli (ETEC:ST) | + | NA | + | + |
| E. coli (ETEC:LT/ST) | + | NA | + | + |

Longevity.

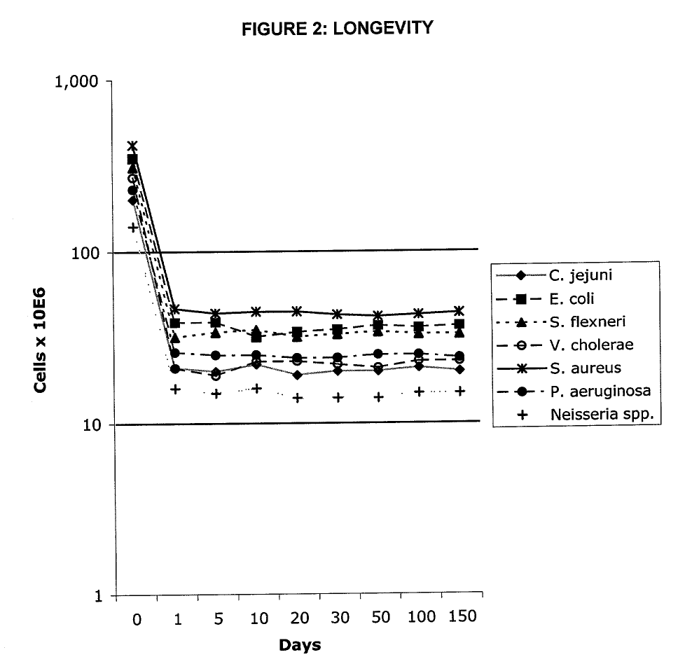

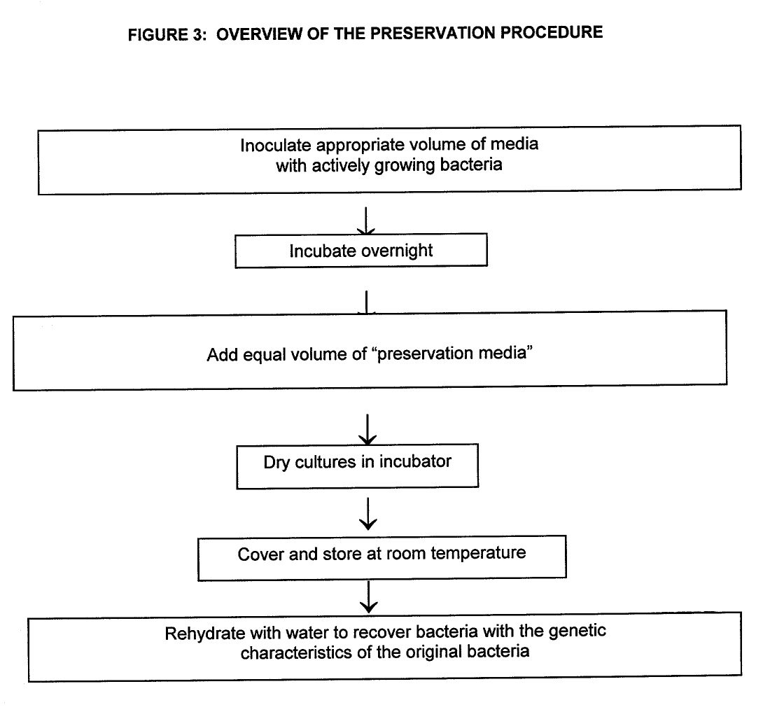

In this set of experiments the longevity of survival of desiccated bacterial strains was evaluated. Briefly, C. jejuni, E. coli, S. flexneri, V. cholerae, S. aureus, P. aeruginosa, and Neisseria spp. were grown overnight in BHI broth at 35° C., 200 RPM. The cells were diluted 1:100 into 5.0 ml of BHI broth in 50 ml conical tube at 35° C., 200 RPM and grown for five hours with sterile distilled water, and 10 μl of the rehydrated cells were serially diluted, then plated onto BHI agar plates and incubated overnight at 35° C. The following day, the number of total colony forming units per ml from each of the wells was determined and the mean recovery calculated. The results of these experiments are summarized in the FIG. 2.

After an initial loss of approximately one order of magnitude, all of the bacterial strains tested were viable through 150 days post-desiccation.

### DISCUSSION & CONCLUSIONS

As shown in FIG. 1, the process entails inoculating bacteria in a small volume of bacterial culture media. The type of media selected is dependent on the species of bacteria to be preserved. Some preparatory procedures are employed prior to preservation. These include: 1) growth of bacteria on suitable media, such as agar or liquid broth; 2) harvesting of organisms, typically by scraping colonies off of agar plates or collection from liquid cultures; 3) mixing the bacterial strains with the preservation solution containing from 10 mM to 200 mM trehalose with or without the addition of 1 to 10 mM one or more of the divalent cations Mg²⁺, Ca²⁺, Zn²⁺, Mn²⁺ (typically in forms such as CaCl₂, ZnCl₂, or MgCl₂). The bacteria in solution are then allowed to dry completely, typically over a period of up to 96 hours. The dried cultures then typically covered covered, sealed in protective bags or containers and stored at room temperature, typically ranging from 20° C. to 25° C. Reconstitution of the desiccated bacteria is accomplished by the addition of water, typically equal to the original total volume and typically pre-warmed to growth temperatures (37° to 42° C.), and then added to bacterial growth media.

Our protocol has the following characteristics and advantages:

1. Desiccation of bacteria in the presence of trehalose and a divalent cation solution permits the survival of the bacteria for prolonged periods.

2. Desiccation can be done at ambient temperatures or within the range of temperatures that are optimal for normal bacterial growth

3. Bacterial loss is at least one order of magnitude after the initial desiccation process.

4. Bacterial loss occurs during the desiccation process and is not affected by the duration of desiccation.

5. Bacteria can be easily reconstituted with a high rate of survival.

6. Bacteria retain characteristics and are not detectably altered after freezing and reconstitution.

This protocol is an effective and economical process for the preservation of bacterial species without requiring special equipment beyond what is typically found in microbiology laboratories. Obviously, many modifications and variations of the present protocol are possible in light of the above results and our findings should be broadly applicable to other bacterial species.

### REFERENCES

1. U.S. Pat. No. 5,059,518 October 1991 Kortright et al . . .

2. U.S. Pat. No. 5,149,653 September 1992 Roser . . .

3. U.S. Pat. No. 5,409,826 June 1993 Maples et al . . .

4. Carpenter, et al., Biochim. Biophys. Acta 923, 109-115, 1987

5. Carpenter, et al., Cyrobiol. 25, 372-376, 1988

6. Crowe and Cooper, Scientific American 225, 30-36, 1971

7. Crowe and Crowe, Devel. Biol. Stand. 74, 285-294, 1991

8. Crowe et al., Ann. Rev. Physiol. 54, 579-599, 1992

9. Hirsh, Cryobiol., 24, 214-228, 1987

10. Israeli et al., Cyrobiol., 30, 519-523, 1993

11. Lee et al., J. Therm. Biol., 17, 263-266, 1992

12. Leslie et al., Biochim. Biophys. Acta, 1192, 7-13, 1994

13. Leslie et al., Appl. Environ. Microbiol. 61, 3592-3597, 1995

14. Mansure et al., Biochim. Biophys. Acta 1191, 309-316, 1994

15. Manual of Clinical Microbiology, 7th Ed., ASM Press, Amer. Soc. of Microbiol., Ed. In Chief, Albert Oliver, et al., Biochim. Biophys. Acta 1267, 92-100, 1995

16. Roser, BioParm, September, 47-53, 1991

17. Shier, Cryobiol. 25, 110-120, 1988

18. Manual of Clinical Microbiology, 6 th Ed., Amer. Soc. of Mircro., Ed. In Chief, Albert Ballows

Obviously, many modifications and variations of the present invention are possible in light of the above teachings. It is therefore to be understood that, within the scope of the appended claims, the invention may be practiced otherwise than as specifically described.

What is claimed is:

1. A dried composition for the preservation of bacteria in a viable state wherein said dried composition comprises: dried viable bacteria and growth medium appropriate for said bacteria wherein said bacteria and growth medium were initially placed in an aqueous solution of 10 mM to 200 mM trehalose and dried at room temperature.

2. The dried composition of claim 1, wherein said bacteria are aerobic.

3. The dried composition of claim 2, wherein said bacteria are selected from the group consisting of Escherichia coli, Pseudomonas aeruginosa, Vibrio cholerae, Salmonella typhimurium, Shigella flexneri, Bacillus subtilis, and Campylobacter jejuni

4. The dried composition of claim 1 further including 1 to 10 mM of a divalent cation.

5. The dried composition of claim 4 wherein the divalent cation is selected from the group consisting of CaCl₂, ZnCl₂, MgCl₂, and combinations of those divalent cations.

6. A dried composition for the preservation of aerobic bacteria in a viable state wherein said dried composition consists essentially of: dried viable aerobic bacteria selected from the group consisting of Escherichia coli, Pseudomonas aeruginosa, Vibrio cholerae, Salmonella typhimurium, Shigella flexneri, Bacillus subtilis, and Campylobacter jejuni and growth medium appropriate for said bacteria wherein said bacteria and growth medium were initially placed in an aqueous solution of 10 mM to 200 mM trehalose and 1 to 10 mM of a divalent cation selected from the group consisting of CaCl₂, ZnCl₂, MgCl₂, and combinations of those divalent cations, and dried at room temperature, wherein said bacteria.

7. A method of preserving bacteria in a viable state at room temperature comprising:
(a) inoculating bacteria in a volume of suitable bacterial culture medium;
(b) growing the inoculated bacteria at its optimal growth temperature to increase volume;
(c) harvesting the grown bacteria;
(d) separating the harvested bacteria from the growth medium;
(e) re-suspending the separated bacteria in fresh growth medium;
(g) incubating the resuspended bacteria at least over-night at incubation temperatures appropriate to the bacteria;
(h) adding to the incubated bacteria an equal volume of a preservation solution comprising an aqueous solution of 10 mM to 200 mM of trehalose;
(i) drying the mixture of incubated bacteria and preservation solution at about 37° C.;
(j) recovering and storing the dried bacteria in sealed containers at room temperature until such time as it is needed; and
(k) restoring the bacteria to its original viable state by rehydration of the dried aerobic bacteria with water.

8. The method of claim 7, wherein said bacteria are aerobic.

9. The method of claim 8, wherein said bacteria are selected from the group consisting of Escherichia coli, Pseudomonas aeruginosa, Vibrio cholerae, Salmonella typhimurium, Shigella flexneri, Bacillus subtilis, and Campylobacter jejuni

10. A method of preserving aerobic bacteria in a viable state at room temperature comprising:
(a) inoculating aerobic bacteria selected from the group consisting of Escherichia coli, Vibrio cholerae, Pseudomonas aeruginosa, Salmonella typhimurium, Shigella flexneri, Bacillus subtilis, and Campylobacter jejuni in a volume of suitable bacterial culture medium;
(b) growing the inoculated bacteria at its optimal growth temperature to increase volume;
(c) harvesting the grown bacteria;
(d) separating the harvested bacteria from the growth medium;
(e) re-suspending the separated bacteria in fresh growth medium;
(g) incubating the resuspended bacteria at least over-night at incubation temperatures appropriate to the bacteria;
(h) adding to the incubated bacteria an equal volume of a preservation solution consisting essentially of an aqueous solution of 10 mM to 200 mM of trehalose and a divalent cation selected from the group consisting of CaCl₂, ZnCl₂, MgCl₂ and combinations of those divalent cations;
(i) drying the mixture of incubated bacteria and preservation solution at about 37° C.;
(j) recovering and storing the dried bacteria in sealed containers at room temperature until such time as it is needed; and
(k) restoring the bacteria to its original viable state by rehydration of the dried aerobic bacteria with water.

11. The method of claim 7 wherein the preservation solution also contains a divalent cation selected from the group consisting of CaCl₂, ZnCl₂, MgCl₂ and combinations of those divalent cations.

12. The dried composition of claim 1, wherein said bacteria is gram-negative.

13. The dried composition of claim 6, where said bacteria is wild-type E. coli.

14. The dried composition of claim 6, where said bacteria is Vibrio cholerae.

15. The dried composition of claim 6, where said bacteria is Salmonella typhimurium.

16. The dried composition of claim 6, where said bacteria is Shigella flexneri 2a

17. The dried composition of claim 6, wherein said bacteria is Bacillus subtilis.

18. The dried composition of claim 6, wherein said bacteria is Campylobacter jejuni.

19. The method of claim 7, wherein said bacteria is gram-negative.

20. The method of claim 10, wherein said bacteria is wild-type E. coli.

21. The method of claim 10, wherein said bacteria is Vibrio cholerae.

22. The method of claim 10, wherein said bacteria is Salmonella typhimurium.

23. The method of claim 10, wherein said bacteria is Shigella flexneri 2a.

24. The method of claim 10, wherein said bacteria Bacillus subtilis.

25. The method of claim 10, wherein said bacteria is Campylobacter jejuni.

26. The method of claim 10, wherein said bacteria is Pseudomonas aeruginosa.
